(19) United States
(12) Patent Application Publication
(10) Pub. No.: US 2009/0314308 A1
(43) Pub. Date: Dec. 24, 2009

(54) Germicidal Floor, Germicidal Foot, And Hand Cleaning System

(76) Inventors: Darrick S.H.L. Kim, Schaumburg, IL (US); John Paoli, Crystal Lake, IL (US)

Correspondence Address: MARSHALL, GERSTEIN & BORUN LLP, 233 SOUTH WACKER DRIVE, 6300 SEARS TOWER, CHICAGO, IL, 60606-6357, US

(21) Appl. No.: 12/109,980

(22) Filed: Apr. 25, 2008

Related U.S. Application Data
(63) Continuation-in-part of application No. 12/101,494, filed on Apr. 11, 2008.
(60) Provisional application No. 60/911,616, filed on Apr. 13, 2007.

(51) Int. Cl. B08B 7/00; A61N 5/06

(52) U.S. Cl. 134/1; 250/492.1

(57) ABSTRACT

A device for simultaneously cleaning hands and feet comprises a first housing and a second housing connected by a pole. An ultraviolet light source and a light reflector is disposed within each of the first and second housings. The ultraviolet light sources and light reflectors are adapted to direct ultraviolet light waves onto the hands and feet of an individual user of the device. The light waves kill or deactivate harmful germs or transmissible diseases disposed on the hands and/or carried by the feet or the socks of the individual, thereby preventing the harmful germs or transmissible diseases from spreading and causing harm to other individuals or animals.

### CROSS-REFERENCE TO RELATED APPLICATIONS

This application is a continuation-in-part of U.S. patent application Ser. No. 12/101,494, filed Apr. 11, 2008, which claims the benefit of priority of U.S. Provisional Patent Application No. 60/911,616, filed Apr. 13, 2007, the entire contents of each of which are hereby incorporated herein by reference.

### BACKGROUND

### Germs

Germs are found all over the world, in all kinds of places. Germs are the cause of many infectious diseases that include pathogenic bacteria, causing diseases such as plague, tuberculosis and anthrax; protozoa, causing diseases such as malaria, sleeping sickness and toxoplasmosis; and also fungi causing diseases such as ringworm, candidiasis or histoplasmosis. Other diseases such as influenza, yellow fever or AIDS are caused by pathogenic viruses, which are not living organisms and are not therefore microorganisms. There are four major types of germs: bacteria, viruses, fungi, and protozoa.

Bacteria are the most diverse and abundant group of unicellular organisms on Earth. Bacteria inhabit practically all environments where some liquid water is available and the temperature is below +140° C. They are found in sea water, soil, air, animals' gastrointestinal tracts, in some cases human body, hot springs and even deep beneath the Earth's crust in rocks. Practically all surfaces which have not been specially sterilized are covered in bacteria. The number of bacteria in the world is estimated to be around five million trillion trillion, or 5×1030. Bacteria are practically all invisible to the naked eye and lack membrane-bound organelles. Some species form extraordinarily resilient spores for survival that under optimal conditions can grow extremely rapidly and can double as quickly as every 10 minutes. Bacteria can reproduce outside of the body or within the body as they cause infections.

Viruses need to be inside living cells to grow and reproduce. Most viruses can't survive very long if they're not inside a living host such as a plant, animal, or person. When viruses get inside people's bodies, they can spread cause diseases. Viruses can't survive, grow, and reproduce unless a person or an animal puts up rental space. Although viruses can only live for a very short time outside other living cells, once they've moved into someone's body, they spread easily and can cause diseases. Viruses are responsible for some minor sicknesses like colds, as well as extremely serious diseases like smallpox or HIV/AIDS.

Fungi are multi-cellular organisms. Unlike other plants, fungi cannot make their own food from soil, water, and air. Instead, fungi get their nutrition from plants, people, and animals. They love to live in damp, warm places, and most fungi are not dangerous. An example of something caused by fungi is athlete's foot, that itchy rash that teens and adults sometimes get between their toes. Fungi are multi-cellular organisms that usually aren't dangerous in a healthy person. Fungi get nutrition from plants, food, and animals in damp, warm environments. Two common fungal infections are athlete's foot and yeast infections. People who have weakened immune systems (from diseases like HIV or cancer) may develop more serious fungal infections.

Protozoa are one-cell organisms that love moisture and often spread diseases through water. Some protozoa cause intestinal infections that lead to diarrhea, nausea, and stomach pain.

### Germs From Shoes

When a person walks outside in a city or suburbs or grass or paved sidewalk or streets or dirt road, shoes pick up a multitude of unwanted hangers-on. These could be:
Remnants of feces from dogs, cats, rodents, birds, other wildlife and sometimes, even humans.
Urine from the same sources.
Excretions such as saliva, mucus, sweat and sometimes, blood or vomit.
Remains from insects and rodents
Remnants of garbage including food waste and toxic cleaning products.

Even indoor spaces such as the floors of restrooms are frequently contaminated with urine and hospitals or doctors' offices are fertile hotspots for a variety of germs that may end up on shoes.

Although these items may be on the ground in very small deposits that are unnoticeable to the naked eye, if those shoes are worn inside a home, there is a risk of spreading these unsanitary matters to carpets and even hard-surfaced floors.

These unsanitary residues from humans, animals, and garbage most likely contain viruses, bacteria, parasites, fungi, and other pathogens that can cause staph infection, hepatitis, herpes, E. coli, tetanus, rabies, strep, hantavirus, cold and flu. And, these germs are brought indoors such as athletic facilities, homes, nurseries, and schools etc and spread from one person to many individuals who come in contact.

### Germs Inside Our Shoes

All of us sweat from the almost quarter of a million sweat glands in each foot. Our feet perspire about a pint of water per day to try to stay cool. However, the sweat cannot evaporate when our feet are smothered inside shoes. What happens to that pint of sweat each foot perspires every day, inside your shoes? It becomes a swamp! It breeds all sorts of harmful bacteria, fungus, and anything any microorganism that is available that wants to blossom! These germs can cause infection. These germs that thrive and multiply in this damp and warm environment produce chemical metabolites that give off foul sour, sweaty, stinky, odor.

After cleaning the feet with soap and water and when the shoes are put back on, you are back to where you left off starting the whole cycle of farming those germs in side the shoes and spreading the germs elsewhere you take off your shoes. This cycle will continue unless all the germs inside the shoes are taken cared of. Therefore, preventing microbial build-up will be the most sensible action one can take from spreading harmful germs. One of the main reasons people dreads taking off shoes at the security checkpoint of an airport is “I do not want to step on a place someone else stepped on because I do not want get that someone else's germs on me and be contaminated.”

If any of the opportunistic pathogens happen to find the right environment inside shoes of a person, now it can become a breeding ground for a disease and infection. Thus, keeping inside of shoes clean of potentially harmful germs is of vital importance for evading contracting and spreading of disease.

### Germs in Gyms

Everyone must agree that one of the most germ-infested places in sports facility is the floor. Most students go to the gym to get healthy, but they may not realize that there are hidden bacterial dangers where they work out. A warm environment full of sweating bodies and exposed skin makes the gym a haven for bacteria and fungi growth. There could be more than 100 million bacteria per square inch on floors. The average gym mat contains 100,000 bacteria per square centimeter, and some contain fecal bacteria (that could causes tummy upsets). Working out in unsupportive and sweaty shoes carries risk of injury and fungal infection. There are 100 times more yeasts and molds in old boots and trainers than in a flushed toilet bowl. The human skin is covered with millions of germs, so when anyone sweats, the germs come pouring off. All of that exposed skin and sweat can create a perfect breeding ground for spreading infections. It's primarily the moisture from the sweat that causes problems by helping germs grow.

Fungi are more complex multi-celled organisms that flourish in places with lots of moisture. Podiatrists also blame dirty exercise mats and floors of sports facility for a rise in athlete's foot and veruccas. Fungus thrives in moist environments, so if they are not kept spotless, mats can be the perfect breeding ground.

Common types include athlete's foot and jock itch. Depending upon the chemical make-up of one's body, certain people are more likely to get one of these fungi. The majority fungal microorganisms are found in the locker rooms of the gym, where people are walking around barefoot. One of the most common diseases lurking in gyms is tinea pedis or athlete's foot (i.e., a fungal skin infection). Its symptoms include cracked, blistered skin along with an itching or burning sensation. If this goes unchecked, it can lead to toenail fungus characterized by blackening of the nails. Onychomycosis is another infectious fungal disease hiding on shower and locker room floors. Its symptoms include yellow, brittle toe- and fingernails.

Sports facility and shared equipment has been linked to the spread of a new type of MRSA in athletic and sports teams. Every workout places is of the risk of the new strains of community-acquired MRSA. An outbreak of MRSA was discovered among football players at Western Carolina University in 2004. It was traced to the locker room, shared towels, equipment and mats. It was found that community-acquired strains of MRSA are quite different from the hospital type. These germs have acquired the ability to produce a potent tissue toxin called Panton-Valentine Leukocidin (PVL), which can lead to skin and soft tissue infections, including flesh-eating forms. MRSA is spread through direct physical contact or indirect touching of contaminated objects. It usually presents itself as some type of skin or soft tissue infection, such as pimples, abscesses or boils.

### Germs at Home

Shoes worn outside of a house carries in as much if not more germs as those worn in an athletic facility. In particular when a household also raises a pet dog or a cat, the risk of exposure to harmful germs rises as these pets venture in and out of the house. No one knows what these pets may have stepped on. It is not difficult to imagine when an individual walks barefoot or even in socks in the house is at risk of contracting highly transmissible disease causing germs through contact on the floor. And we drop and touch objects on the floor, pick them up with hands, and eat with the hands without washing, transporting the germs from the floor and other infested places to the mouth. Threadworm parasites and infections such as diarrhea and tummy upsets can be transmitted through contact with dirty floor. Thus it is a good suggestion to sanitize one's feet now and then from harmful germs that may have gotten on from the floor now and then, in particular when the individual is at risk of infection due to lowered immune conditions such as diabetes, AIDs, lupus, scratch or punctured wounds on the feet etc . . . .

### Hygiene/Protection From Germs

Most germs are spread through the air in sneezes, coughs, or even breaths. Germs can also spread in sweat, saliva, and blood. Some pass from person to person by touching something that is contaminated, like shaking hands with someone who has a cold and then touching your own nose. So the best way to protect from germs is to steer clear of the things that can spread them.

Hygiene is the avoidance of infection or food spoiling by eliminating or reducing harmful germs to acceptable levels. This include washing with soap and water or applying sanitizing lotions or wiping with sanitizing tissue/cloth applied with sanitizing lotions that contain alcohols or antibiotics which can eliminate germs. But often times sanitizing hands using soap and water can be cumbersome or not readily available.

### Staph Infection and Germs in Hospitals and MRSA

Staph infection is most commonly caused by Staphylococcus aureus that is a spherical bacterium, frequently living on the skin or in the nose of a person, that can cause a range of illnesses from minor skin infections, such as pimples, impetigo, boils, cellulitis and abscesses, to life-threatening diseases, such as pneumonia, meningitis, endocarditis, Toxic shock syndrome (TSS), and septicemia. Methicillin-resistant Staphylococcus aureus (MRSA) belongs to the large group of bacteria known as Staphylococci, often referred to as Staph. About 25%-30% of all people have Staph within the nose, but it normally does not cause an infection. In contrast, only about 1% of the population has MRSA. Since the 1960s, hospitals and other institutional health-care settings, such as nursing homes, where they tend to strike older people, those who are very ill, and people with a compromised immune system have been battling MRSA infection. In health-care settings, MRSA is a frequent cause of surgical wound infections, urinary tract infections, bloodstream infections (sepsis), and pneumonia. MRSA outbreaks, however, are appearing increasingly in the community where people who have not been hospitalized or had a medical procedure performed in the past year, and who do not have immune deficiency. These infections are termed community-associated MRSA infections (CA-MRSA). The U.S. Centers for Disease Control and Prevention (CDC) estimates that about 12% of MRSA infections are now community-associated, although this percentage can vary by community and patient population. Recent reports show that CA-MRSA is finding new homes in the community, particularly among kids. Over the past month, schools have shuttered their doors, sanitized their hallways, and alerted parents to the presence of the spreading drug-resistant bacteria in locker rooms and on wrestling mats. At least three students have died of the infection. Last month Center for Disease Control (CDC) and prevention released a report on invasive MRSA that in 2005, there were 32 cases per 100,000 people in the U.S. and that 14% of these occurred without previous visitation to hospitals and institutional health-care settings.

Methicillin-resistant Staphylococcus aureus (MRSA) may also be known as:

multiple-resistant Staphylococcus aureus

oxacillin-resistant Staphylococcus aureus (ORSA)

community-acquired MRSA (CA-MRSA)

hospital-acquired MRSA (HA-MRSA)

### Germicidal Ultraviolet Light:

### What is Ultraviolet?

Ultraviolet light is part of the light spectrum, which is classified into three wavelength ranges:

UV-C, from 100 nanometers (nm) to 280 nm

UV-B, from 280 nm to 315 nm

UV-A, from 315 nm to 400 nm

### What is Germicidal Ultraviolet?

UV-C light is germicidal—i.e., it deactivates the DNA of bacteria, viruses and other pathogens and thus destroys their ability to multiply and cause disease. Specifically, UV-C light causes damage to the nucleic acid of microorganisms by making them form covalent bonds between certain adjacent bases in the DNA. The formation of such bonds prevents the DNA from being unzipped for replication, and the organism is unable to reproduce. In fact, when the organism tries to replicate, it dies. UVC has extremely low penetrating ability and does not penetrate past the dead-cell layers of the skin. UV will cause eye irritations or burns after prolonged exposure.

### What are the Beneficial Uses of Germicidal UV?

Ultraviolet technology is a non-chemical approach to disinfection. In this method of disinfection, nothing is added which makes this process simple, inexpensive and requires very low maintenance. Ultraviolet purifiers utilize germicidal lamps that are designed and calculated to produce a certain dosage of ultraviolet (usually at least 16,000 microwatt seconds per square centimeter but many units actually have a much higher dosage.) The principle of design is based on a product of time and intensity—you must have a certain amount of both for a successful design. Germicidal UV has been used in water disinfection systems for many years. As stated by the American Water Works Association, “ . . . UV light disinfection process does not use chemicals. Microorganisms, including bacteria, viruses, and algae, are inactivated within seconds of UV light disinfection. Ultraviolet is effective in inactivating Cryptosporidium, while at the same time decreasing chlorinated disinfection by-products . . . U.S. cities currently using or planning to use UV light water disinfection include: Seattle, Washington, New York City, Central Lake County, Illinois, Albany, New York, Tempe, Arizona . . . ”

### Here are Just a Few of the Current Applications:

### Drinking Water

under sink installs & water vending machines
aircraft, boats & recreational vehicles
water wells & water cisterns
swimming pool & hot tubs
farms, ranches & trailer parks
schools & hotels
aquarium, hatcheries and nurseries
ice making Food Processing
brewery & winery
soft drinks, fruit drinks and juices
bottling facilities
diary processing
liquid sugars, sweeteners and edible oils
water based lubricants
pure wash water

### Medical

pharmaceutical production
laboratories, hospitals and clinics
maternity labor and delivery areas
pathology labs, kidney dialysis
animal husbandry Industries
cosmetics and electronic production
pond & lake reclamation
laundry water

Numerous health studies have shown that germicidal ultraviolet light—UVC—is very effective against allergies, asthma, mold, mildew, fungi and any DNA based airborne viruses, bacteria and spores. UV light can play a great role in secondary allergy prevention especially where airborne allergens are concerned. It can reduce suffering from allergies. The UV air cleaners help control germs that make asthma worse. Ultraviolet Germicidal Irradiation—UVGI is the use of short UV waves, known as UVC, which has been proven to kill or inactivate tuberculosis in the indoor air. There are two ways in which UV lamps are used for indoor air quality control—upper-room air and in-duct ultraviolet germicidal irradiation. Many airborne pathogens, such as Anthrax and its spores, can be removed from the indoor air by the use of germicidal ultraviolet air purifiers.

Germicidal UV light (UVC) has been successfully utilized in hospitals and public buildings to inhibit microbial growth, spreading of infections and to increase the indoor air quality. UVC can also help against mold problems by rendering airborne mold particles and mold spores non-viable and even sterilizing surface mold colonies if they are directly irradiated with germicidal UV (UVC) light. Even the dead mold spores and mold particles can be allergenic or toxic so it is strongly recommended to always use germicidal UV light in conjunction with a HEPA filtration system.

### How Does Ultraviolet Light Work?

Germicidal ultraviolet lamp is a short wave low pressure mercury vapor tubes that produces ultraviolet wavelengths that are lethal to micro-organisms. Approximately 95% of the ultraviolet energy emitted is at the mercury resonance line of 254 nanometers. This wavelength is in the region of maximum germicidal effectiveness and is highly lethal to virus, bacteria and mold spores. Therefore, the genetic material of the micro-organism that is exposed to the germicidal uv light and is deactivated, which prevents them from reproducing and renders them harmless.

Germicidal UV light has been successfully utilized in hospitals and public buildings to inhibit microbial growth, spreading of infections and to increase indoor air quality. UVC can also help against mold problems for mold remediation and mold inhibition by rendering airborne mold particles and mold spores non-viable and even sterilizing surface mold colonies if they are directly irradiated with UVC light. These American Air & Water resources show mold/mildew irradiation dosages and indoor air testing results for mold and mold spores:

The following external resources present more information on mold/mildew cleaning and different mold remediation tips:

Please note that many variables take place in a real world environment that makes actual calculating of the UV dosage very difficult (air flow, humidity, distance of microorganism to the UV light and time). However, it is proven that UV light will kill any DNA-based organism given enough UV dosage and that UV light breaks down DNA on a cumulative basis. The UV light helps to reduce incidences of inhaled pathogens for persons who reside or work in indoor environments.

The following are incident energies of germicidal ultraviolet radiation at 253.7 nanometers necessary to inhibit colony formation in microorganisms (90%) and for complete destruction:

| | Energy dosage of Ultraviolet | |
|---|---|---|
| | radiation in μW/cm² needed | |
| Organisms: | for kill factor | |
| Bacteria | 90% | 100% |
| Bacillus anthracis - Anthrax | 4,520 | 8,700 |
| Bacillus anthracis spores - Anthrax spores | 24,320 | 46,200 |
| Bacillus magaterium sp. (spores) | 2,730 | 5,200 |
| Bacillus magaterium sp. (veg.) | 1,300 | 2,500 |
| Bacillus paratyphusus | 3,200 | 6,100 |
| Bacillus subtilis spores | 11,600 | 22,000 |
| Bacillus subtilis | 5,800 | 11,000 |
| Clostridium tetani | 13,000 | 22,000 |
| Corynebacterium diphtheriae | 3,370 | 6,510 |
| Ebertelia typhosa | 2,140 | 4,100 |
| Escherichia coli | 3,000 | 6,600 |
| Leptospiracanicola - infectious Jaundice | 3,150 | 6,000 |
| Microccocus candidus | 6,050 | 12,300 |
| Microccocus sphaeroides | 1,000 | 15,400 |
| Mycobacterium tuberculosis | 6,200 | 10,000 |
| Neisseria catarrhalis | 4,400 | 8,500 |
| Phytomonas tumefaciens | 4,400 | 8,000 |
| Proteus vulgaris | 3,000 | 6,600 |
| Pseudomonas aeruginosa | 5,500 | 10,500 |
| Pseudomonas fluorescens | 3,500 | 6,600 |
| Salmonella enteritidis | 4,000 | 7,600 |
| Salmonela paratyphi - Enteric fever | 3,200 | 6,100 |
| Salmonella typhosa - Typhoid fever | 2,150 | 4,100 |
| Salmonella typhimurium | 8,000 | 15,200 |
| Sarcina lutea | 19,700 | 26,400 |
| Serratia marcescens | 2,420 | 6,160 |
| Shigella dyseteriae - Dysentery | 2,200 | 4,200 |
| Shigella flexneri - Dysentery | 1,700 | 3,400 |
| Shigella paradysenteriae | 1,680 | 3,400 |
| Spirillum rubrum | 4,400 | 6,160 |
| Staphylococcus albus | 1,840 | 5,720 |
| Staphylococcus aerius | 2,600 | 6,600 |
| Staphylococcus hemolyticus | 2,160 | 5,500 |
| Staphylococcus lactis | 6,150 | 8,800 |
| Streptococcus viridans | 2,000 | 3,800 |
| Vibrio comma - Cholera | 3,375 | 6,500 |
| Molds | 90% | 100% |
| Aspergillius flavus | 60,000 | 99,000 |
| Aspergillius glaucus | 44,000 | 88,000 |
| Aspergillius niger | 132,000 | 330,000 |
| Mucor racemosus A | 17,000 | 35,200 |
| Mucor racemosus B | 17,000 | 35,200 |
| Oospora lactis | 5,000 | 11,000 |
| Penicillium expansum | 13,000 | 22,000 |
| Penicillium roqueforti | 13,000 | 26,400 |
| Penicillium digitatum | 44,000 | 88,000 |
| Rhisopus nigricans | 111,000 | 220,000 |
| Protozoa | 90% | 100% |
| Chlorella Vulgaris | 13,000 | 22,000 |
| Nematode Eggs | 45,000 | 92,000 |
| Paramecium | 11,000 | 20,000 |
| Virus | 90% | 100% |
| Bacteriopfage - E. Coli | 2,600 | 6,600 |
| Infectious Hepatitis | 5,800 | 8,000 |
| Influenza | 3,400 | 6,600 |
| Poliovirus - Poliomyelitis | 3,150 | 6,600 |
| Tobacco mosaic | 240,000 | 440,000 |
| Yeast | 90% | 100% |
| Brewers yeast | 3,300 | 6,600 |
| Common yeast cake | 6,000 | 13,200 |
| Saccharomyces carevisiae | 6,000 | 13,200 |
| Saccharomyces ellipsoideus | 6,000 | 13,200 |
| Saccharomyces spores | 8,000 | 17,600 |

### UV Tests

Tests conducted by Light Sources Inc—Orange, Conn. and verified by American Ultraviolet Company—Lebanon, Ind. revealed that American-Lights® produces 800 microwatts@1 foot with 534 feet per minute air flow@55° F. To compute time needed to sterilize germs in the following chart at 1 foot distance from the light, divide the dosage required by 800. Example: for 90% kill factor of Bacillus subtilis spores: 11,600 divided by 800=14.5 seconds.

Microorganisms, including bacteria, viruses, and algae, are inactivated within seconds of UV light disinfection, but all are not equally sensitive. Generally, viruses and algae are more resistant to disinfection by UV light.

UV light is effective in inactivating Cryptosporidium, while at the same time decreasing chlorinated disinfection by-products.

UV disinfection is used in air and water purification, sewage treatment, protection of food and beverages, and many other disinfection and sterilization processes.

### Infectious Disease

An infectious disease is a clinically evident disease of humans or animals that damages or injures the host so as to impair host function, and results from the presence and activity of one or more pathogenic microbial agents, including viruses, bacteria, fungi, protozoa, multicellular parasites, and aberrant proteins known as prions. Transmission of an infectious disease may occur through several pathways; including through contact with infected individuals, by water, food, airborne inhalation, or through vector-borne spread.

A contagious disease (also called a communicable disease) is an infectious disease that is capable of being transmitted from one person or species to another. Contagious diseases are often spread through direct contact with an individual, contact with the bodily fluids of infected individuals, or with objects that the infected individual has contaminated.

The term infectivity describes the ability of an organism to enter, survive and multiply in the host, while the infectiousness of a disease indicates the comparative ease with which the disease is transmitted to other hosts. An infection however, is not synonymous with an infectious disease, as an infection may not cause clinical symptoms or impair host function.

### Classification

Among the almost infinite varieties of microorganisms, relatively few cause disease in otherwise healthy individuals. Infectious disease results from the interplay between those few pathogens and the defenses of the hosts they infect. The appearance and severity of disease resulting from any pathogen depends upon the ability of that pathogen to damage the host as well as the ability of the host to resist the pathogen. Infectious microorganisms, or microbes, are therefore classified as either primary pathogens or as opportunistic pathogens according to the status of host defenses.

Primary pathogens cause disease as a result of their presence or activity within the normal, healthy host, and their intrinsic virulence (the severity of the disease they cause) is, in part, a necessary consequence of their need to reproduce and spread. Many of the most common primary pathogens of humans only infect humans, however many serious diseases are caused by organisms acquired from the environment or which infect non-human hosts.

Organisms which cause an infectious disease in a host with depressed resistance are classified as opportunistic pathogens. Opportunistic disease may be caused by microbes that are ordinarily in contact with the host, such as bacteria or fungi in the gastrointestinal or the upper respiratory tract, and they may also result from (otherwise innocuous) microbes acquired from other hosts (as in Clostridium difficile enterocolitis) or from the environment as a result of traumatic introduction (as in surgical wound infections or compound fractures). An opportunistic disease requires impairment of host defenses, which may occur as a result of genetic defects (such as Chronic granulomatous disease), exposure to antimicrobial drugs or immunosuppressive chemicals (as might occur following poisoning or cancer chemotherapy), exposure to ionizing radiation, or as a result of an infectious disease with immunosuppressive activity (such as with measles, malaria or HIV disease). Primary pathogens may also cause more severe disease in a host with depressed resistance than would normally occur in an immunosufficient host.

### Transmission

An infectious disease is transmitted from some source. Defining the means of transmission plays an important part in understanding the biology of an infectious agent, and in addressing the disease it causes. Transmission may occur through several different mechanisms. Respiratory diseases and meningitis are commonly acquired by contact with aerosolized droplets, spread by sneezing, coughing, talking or even singing. Gastrointestinal diseases are often acquired by ingesting contaminated food and water. Sexually transmitted diseases are acquired through contact with bodily fluids, generally as a result of sexual activity. Some infectious agents may be spread as a result of contact with a contaminated, inanimate object (known as a fomite), such as a coin passed from one person to another, while other diseases penetrate the skin directly.

Transmission of infectious diseases may also involve a “vector”. Vectors may be mechanical or biological. A mechanical vector picks up an infectious agent on the outside of its body and transmits it in a passive manner. An example of a mechanical vector is a housefly, which lands on cow dung, contaminating its appendages with bacteria from the feces, and then lands on food prior to consumption. The pathogen never enters the body of the fly.

How Some Infectious Diseases Are Spread
Method of Transmission

| | Respiratory Transmission | Fecal-Oral Transmission | |
|---|---|---|---|
| Direct Contact | (passing from the lungs, | (touching feces or | |
| with infected | throat, or nose of one | objects contaminated | |
| person's | person to another person | with feces then | Blood |
| skin or body fluid | through the air) | touching your mouth) | Transmission |
| Chickenpox* | Chickenpox* | Campylobacter** | Cytomegalovirus |
| Cold Sores | Common Cold | E. Coli O157** | Hepatitis B* |
| Conjunctivitis | Diphtheria | Enterovirus | Hepatitis C |
| Head Lice | Fifth Disease | Giardia | HIV Infection |
| Impetigo | Bacterial meningitis* | Hand-Foot-Mouth | |
| Ringworm | Hand-Foot-Mouth | Disease | |
| Scabies | Disease | Hepatitis A* | |
| | Impetigo | Infectious Diarrhea | |
| | Influenza* | Pinworms | |
| | Measles* | Polio* | |
| | Mumps* | Salmonella** | |
| | Pertussis* | Shigella | |
| | Pneumonia | | |
| | Rubella* | | |

*Vaccines are available for preventing these diseases.
**Often transmitted from infected animals through foods or direct contact.

As the table shows,

Skin infections may be spread by touching fluid from another person's infected sores.

Respiratory-tract infections with symptoms such as coughs, sneezes, and runny noses are spread mainly through exposure to fluids present in or expelled from another person's mouth and throat (saliva or mucus), often when an uninfected person touches these discharges with their hands and then touches their mouth, eyes, or nose.

Intestinal tract infections, including some types of diarrhea, usually are spread through exposure to germs in the feces. Many of the germs discussed in this manual are spread through what is known as “fecal-oral” transmission. This means that germs leave the body of the infected person in the feces (poop) and enter the body of another person through the mouth. In most situations, this happens when objects (including toys, fingers, or hands) which have become contaminated with undetectable amounts of feces are placed in the mouth. Fecal-oral transmission can also occur if food or water is contaminated with undetectable amounts of human or animal feces, and then is eaten or drunk. Improperly prepared foods made from animals (for example, meat, milk, and eggs) are often the source of infection with Campylobacter, E. coli O157, and Salmonella.

Some infections, like infection with Salmonella and Campylobacter, may be spread through direct exposure to infected animals.

Blood infections are spread when blood (and sometimes other body fluids) from a person with an infection gets into the bloodstream of an uninfected person. This can happen when infected blood or body fluid enters the body of an uninfected person through cuts or openings in the skin; the mucous membrane that lines body cavities, such as the nose and eye; or directly into the bloodstream, as with a needle.

Some diseases, such as chickenpox, impetigo, and hand-foot-and mouth disease, can have more than one transmission route. For example, they may be spread through air or by direct contact with the infectious germ.

### List of Infectious Diseases That May Be of Concern:

### Viral Infectious Diseases

AIDS-AIDS Related Complex-Chickenpox (Varicella)-Common cold-Cytomegalovirus Infection-Dengue fever-Ebola haemorrhagic fever-Hand, foot and mouth disease-Hepatitis-Herpes simplex-Herpes zoster-HPV-Influenza (Flu)-Lassa fever-Measles-Marburg haemorrhagic fever-Infectious mononucleosis-Mumps-Poliomyelitis-Progressive multifocal leukencephalopathy-Rubella-Smallpox (Variola)-Viral encephalitis-Viral gastroenteritis-Viral meningitis-Viral pneumonia-West Nile disease-Yellow fever

### Bacterial Infectious Diseases

Anthrax-Bacterial Meningitis-Botulism-Brucellosis-Campylobacteriosis-Cat Scratch Disease-Cholera-Diphtheria-Epidemic Typhus-Gonorrhea-Impetigo-Legionellosis-Leprosy (Hansen's Disease)-Leptospirosis-Listeriosis-Melioidosis-MRSA infection-Plague-Pneumococcal pneumonia-Psittacosis-Q fever-Rocky Mountain Spotted Fever (RMSF)-Salmonellosis-Scarlet Fever-Shigellosis-Syphilis-Tetanus-Trachoma-Tuberculosis-Tularemia-Typhoid Fever-Typhus; Urinary Tract Infections

### Parasitic Infectious Diseases

African trypanosomiasis-Amebiasis-Ascariasis-Babesiosis-Chagas Disease-Clonorchiasis-Cryptosporidiosis-Cysticercosis-Diphyllobothriasis-Dracunculiasis- Echinococcosis-Enterobiasis-Fascioliasis-Fasciolopsiasis-Filariasis-Free-living amebic infection-Giardiasis-Gnathostomiasis-Hymenolepiasis-Isosporiasis-Kala-azar- Leishmaniasis-Metagonimiasis-Myiasis-Onchocerciasis-Pediculosis-Pinworm Infection-Scabies-Taeniasis-Toxocariasis-Toxoplasmosis-Trichinellosis-Trichinosis-Trichuriasis-Trichomoniasis-Trypanosomiasis

### Fungal Infectious Diseases

Aspergillosis-Blastomycosis-Candidiasis-Coccidioidomycosis-Cryptococcosis-Histoplasmosis-Tinea pedis

Antibiotics and Resistance. Antibiotics are compounds that are produced by microorganisms that kill or inhibit the growth of other microorganisms. Those that kill bacteria are called bactericidal; those that prevent multiplication (and rely on the body's defense mechanisms to deal with the limited number of living organisms) are called bacteriostatic. Some antibiotics are effective against a limited number of microorganisms, others may have more widespread effect.

Because microorganisms are continually in a state of evolution, strains may evolve that are resistant to a particular antibiotic. In addition, resistance characteristics can be transferred from some microorganisms to others (this is particularly true of organisms that inhabit the gastrointestinal tract). The likelihood that resistance will develop is increased if antibiotics are used in an indiscriminate manner and in inadequate amounts (either in terms of individual dosage or in length of therapy). Antimicrobial resistance is a growing problem: organisms that once were exquisitely sensitive to a particular antibiotic may now have developed significant (or total) resistance to it. This necessitates either increasing the dose of the antibiotic administered (in the case of partial resistance) or developing totally new drugs to treat the infection (in the case of total resistance). A few microorganisms (such as enterococcus, an organism that lives in the intestinal tract and is particularly likely to cause infections in gravely ill patients with compromised immune systems) have developed such widespread resistance that it is a real challenge to treat them effectively, resulting in a need to develop even more antibiotics.

### Molds

Different types of mold—black mold, toxic mold, allergenic mold—are present all the time around us and in the air we breathe. In low levels, molds and mold spores are generally harmless but if their levels increase they can affect people; especially people with allergies, asthma and respiratory conditions or suppressed immune system.

Allergenic mold and mold spores are normally not dangerous to humans in low amounts, but they cause allergic or asthmatic symptoms. Generally, these types of mold can be relatively easy and safely cleaned and removed. Note that even dead mold spores or mold particles can trigger allergy symptoms or allergic reactions.

Mycotoxic mold and mold spores are those containing toxins in the cell wall. These types of mold can cause serious health problems in humans and animals. These molds range from short-term irritation to immunosuppression, to cancer and even death. If toxic molds are identified, it is suggested that you seek advice from an Industrial Hygienist or other mold professional for guidance. The average homeowner should NOT attempt the abatement of these types of mold.

Pathogenic mold is the type of mold that causes infections. Pathogenic molds can cause serious health effects in persons with suppressed immune systems, those taking chemotherapy, and those with HIV/AIDS, or autoimmunity disorders. If any pathogenic molds are identified, it is suggested that you seek advice from an Industrial Hygienist or other mold professional. The average homeowner should NOT attempt the abatement of this type of molds.

Hyphae & hyphal elements are single, unidentifiable fragments of mold. Although they might not be traceable to a specific mold species, these fragments can be responsible for allergic reactions in some people and may indicate previous or current growth. Ascospores and basidiospores are clusters of spores that may not be easily identified as a specific species, but may represent a mold problem in the property.

### Description of Infectious Diseases and Conditions That Directly Affect Feet

Foot infections can be difficult problems for physicians to treat due to the biomechanical complexities of the extremity and the underlying circumstances that cause the infections. Typically, they follow a traumatic event or tissue loss with contamination by foreign materials and/or colonization by bacteria. When a healthy patient or one without metabolic or peripheral vascular disease (PVD) presents with pedal infections, a traumatic process usually is involved. However, the more common presentation is that of a patient whose health is compromised with a metabolic or peripheral vascular defect that complicates optimum successful treatment.

Treatment strategies for foot infections have been changing and evolving as a result of pharmacologic and technical breakthroughs. Plastic and reconstructive techniques for limb salvage have altered the course of treatment for foot infections, with a goal towards functional restoration and a major decrease in amputation rates.

Physicians need to be aware of the many different types of foot infections that exist. Some foot infections are very simple, and others are quite complex. They are categorized into 3 groups including soft tissue, bone, and those associated with patients with diabetes. Foot infections in persons with diabetes can be unpredictable and are typically polymicrobial; therefore, they are discussed in their own venue.

Soft tissue infections of the foot consist of any infectious process affecting the skin, subcutaneous tissue, adipose tissue, superficial or deep fascia, ligaments, tendons, tendon sheaths, joints, and/or joint capsules. Considering that there are more than 20 joints, 44 tendons, approximately 100 ligaments, 4 major compartments, and numerous fascial planes in the normal foot, one can easily recognize the potential for complex problems.

Many events can be responsible for these soft tissue infections. A description of soft tissue infections includes simple, moderate, and severe infections, which includes but is not limited to the immunocompromised patient, infections associated with PVD, emergency soft tissue infections, and infections associated with trauma.

Bone commonly is involved when any type of infectious process is present in the foot. Bone is predisposed to this because of its close proximity to the skin and lack of a thick, soft tissue, protective layer throughout most parts of the foot. This process with its diagnostic and treatment strategies is described in detail in this article.

Diabetes mellitus (DM) has been diagnosed in approximately 14 million US citizens. It can produce a complex clinical picture due to its involvement in numerous different organ systems. The combination of diabetic peripheral neuropathy and compromised distal vascularity act synergistically putting these patients at high risk for pedal complications. Individuals with diabetes tend to develop ulcerations in the feet, which often lead to infection of the soft tissue and bones.

### Athlete's Foot

A group of mold-like fungi called dermatophytes causes athlete's foot. These organisms sprout tendril-like extensions that infect the superficial layer of the skin. In response to this fungal growth, the basal layer of the skin produces more skin cells than usual. As these cells push to the surface, the skin becomes thick and scaly. Most often, the more the fungi spread, the more scales your skin produces, causing the ring of advancing infection to form.

“Athlete's foot causes dry, scaly skin, particularly between the toes, and can lead to splits in the heel. “As fingernails are one of the dirtiest parts of the body, I've seen serious secondary bacterial infections as a result of scratching and breaking the skin.”

### Cellulitis

Cellulitis is often the first sign of a soft tissue infection of the foot. In most cases, this marker represents an isolated localized skin infection but may represent a more severe process. Cellulitis usually originates from minor cuts and abrasions but also comes from more severe puncture wounds or trauma. Group A streptococci is the most common bacterial contaminant responsible for soft tissue infections, and Staphylococcus aureus is the second most common. Each is present in natural skin flora.

Initial treatment for simple cellulitis as a result of an abrasion in a host who is not immunocompromised includes oral antibiotics using first-generation cephalosporins, aminopenicillins, or quinolones. Group A streptococci is the most common pathogen and is usually susceptible to penicillin V and cephalexin. In more severe cases, oxacillin 2 g administered intravenously every 4 hours or cefazolin 1 g administered intravenously every 8 h can be used. Simple cellulitis usually responds well to antibiotics, rest, and elevation of the extremity. However, in more severe cases invasive treatment with debridement of necrotic tissue becomes necessary if septic embolization ensues.

### Paronychia

Paronychia is a more common soft tissue infection with inflammation of the periungual area adjacent to the nail grooves and borders. It can be initiated by a traumatic event such has dropping objects on the toes or having them stepped on; more often, paronychia results from an ingrown toenail (onychocryptosis). Underlying onychomycosis also can be a predisposing factor, which results in paronychia.

Initial treatment should include antibiotic therapy and warm soaks to the affected digit. Antibiotic therapy should be directed toward the offending pathogens, which are commonly skin flora. When onychocryptosis is the underlying etiology, that portion of the nail should be removed to address the soft tissue reaction. A partial nail avulsion involves removing the border of the ingrown nail. Chronic recurrent paronychias can be treated in a surgical manner both chemically and via local excision.

### Puncture Wounds

Puncture wounds occur more than 50% of the time on the planter surface of the foot with more than 90% of these involving penetration of a nail. Other objects in this category include wood, metal, plastic, glass, and animal and human bites. Puncture wounds have the potential to inoculate deep spaces of the foot, including bones, joints, tendons, and deep fascia, and serious complications can arise. Therefore, the depth of penetration is one of the most important factors in determining if a wound will resolve without complex intervention. Degree of infection can depend on location, type of penetrating object, retained foreign bodies from pieces breaking off, and penetration through shoes and socks.

The most common organisms implicated in penetrating wounds are S aureus, beta-hemolytic streptococci, and various anaerobic bacteria. Pasteurella multocida is typical in dog and cat bites or claw puncture wounds. Viridans streptococcus is responsible for most problems related to human bites. Pseudomonas aeruginosa is usually responsible for infection when the injury is due to object penetration through shoes and socks.

### Immunocompromise

Pedal infections in patients who are immunocompromised can be difficult to diagnose and treat due to comorbidities, which often alter the presentation and require treatment. This group of patients includes persons with HIV, systemic lupus erythematosus (SLE), rheumatoid arthritis (RA) and high-dose corticosteroid use, DM, and asplenia. These patients can have impaired host defenses and are at higher risk of acquiring infections.

### Human Immunodeficiency Virus

Patients with HIV are more susceptible to fungal and viral pedal infections. Tinea pedis and onychomycosis often are observed in this population. Human papilloma virus, manifesting itself as verruca plantaris, occurs at a higher rate than in the normal population. Fungal infections usually can be managed with a variety of antifungal creams. Verrucae can be more challenging to treat. The condition often requires several modalities for complete resolution. Common therapies include salicylic acid application, cryotherapy, blistering agents, and surgical excision.

### Systemic Lupus Erythematosus

Individuals with SLE are predisposed to infection due to their impaired humoral immunity and lowered T-lymphocyte-mediated immunity, which results from immunosuppressive therapy. Lupus flares can be mistaken for an infection and the differential diagnosis must be evaluated carefully. SLE skin and soft tissue infections most commonly are caused by S. aureus and less commonly by group A streptococci. The absence of a true leukocytosis may create a confusing environment for the diagnostician.

### Rheumatoid Arthritis

Patients with systemic arthropathies such as RA are often on long-term corticosteroid therapy, which suppresses cell-mediated immunity; thus, a higher risk of infection results. Classic presentations include synovitis, pannus formation, and periarticular bone and cartilage destruction. These local manifestations of systemic disease can be confused with soft tissue infection. Approximately 20% of patients with RA have rheumatoid nodules. They frequently occur over pressure areas, which tend to rupture in the foot causing skin breakdown, erythema, and infection. Therefore, patients with RA presenting with possible pedal infections require a thorough workup to exclude the conditions discussed above.

### Diabetes

Persons with diabetes are truly compromised due to their impaired host defenses, decreased perfusion to the lower extremities, and diminished sensation in the feet secondary to peripheral neuropathy.

### Asplenia

The spleen is the primary site for immunoglobulin M (IgM) synthesis, which is the first early response of the body. This places patients with asplenia at higher risk for all infections. They are at a particular high risk for infection with encapsulated bacterial organisms. It is important to determine if they have joint implants or internal fixation in the feet because of the organisms' affinity for seeding these areas. For fractures requiring open reduction and external fixation (ORIF), prophylaxis against these pathogens is appropriate. Typical pedal infections in these patients are clinically indistinguishable from those in healthy hosts, except that they are often more severe.

Antibiotic coverage should be directed towards the encapsulated pathogens and consist of third- or fourth-generation cephalosporins. Imipenem should be used in the patient who is allergic to penicillin. If hardware is present and has become colonized, it should be removed for complete resolution of the infection.

### Peripheral Vascular Disease

The poorly perfused or ischemic foot is prone to more frequent and severe infections due to low oxygen tension. The inflammatory response to stress can be reduced. Decreased local perfusion, edema formation, and neutrophil infiltration are present. Small abrasions in these patients can become quite problematic because the lesions do not have sufficient blood supply to heal properly. Prolonged healing time leaves the patient more susceptible to infection and complications. Ischemic feet are also prone to ischemic-type ulcerations that have an extended healing time and an increased chance of infection.

### Necrotizing Fasciitis

Necrotizing fasciitis is characterized by widespread necrosis of fascia and deeper subcutaneous tissues, with initial sparing of skin and muscle. Eventually, skin involvement is noted, with cellulitis evolving into cutaneous gangrene. The most common underlying risk factor is being a patient with DM. One recent study by Elliott reported that foot ulcerations and infections associated with diabetes were the second most common cause of necrotizing fasciitis; thus, 15.2% of cases of necrotizing fasciitis are due to foot ulcerations and infections associated with diabetes.

Surgical wounds and infections resulting from intravenous drug abuse or “skin popping” also can lead to necrotizing infections. Aerobic streptococci are typical pathogens in addition to Bacteroides species, staphylococci, and enterococci, which all play a role in the infectious process. E coli and Proteus species are facultative anaerobic gram-negative rods that often are cultured from these wounds.

### Gas Gangrene

Gas gangrene or clostridial myonecrosis is considered a surgical emergency. It includes a rapid fulminating course, severe toxin-related systemic toxicity, a vast level of tissue destruction, and a high mortality rate. Rapid diagnosis and aggressive management are crucial with respect to limb preservation.

Six different species of Clostridia can be responsible for the soft tissue destruction; however, Clostridium perfringens is the most common. C perfringens produces 12 active tissue toxins responsible for the syndromes of gas gangrene. Clostridia organisms are saprophytes and are quite ubiquitous. Infections leading to gas gangrene require an opportunistic environment. Prerequisites include a wound, contamination with Clostridia organisms, and a depressed oxygen state at the site of inoculation. This accounts for the increased incidence of gangrene noted in patients with diabetes and patients with PVD. The decreased oxygen state also can be observed poleoperatively from local edema and dressings.

### Chronic Osteomyelitis

Chronic osteomyelitis is defined as the presence of bone infection for more than 6 weeks. In the foot, it is most commonly observed in the diabetic population because their compromised immunity and vascular insufficiency predispose them to deep bone infections. Osteomyelitis also can be associated with open fractures, PVD, immuno-compromised hosts, and improper treatment of the acute condition.

### SUMMARY OF THE DISCLOSURE

One embodiment of the present disclosure comprises a first housing, at least one ultraviolet light source and light reflector, and one of a motion detector or a switch. The first housing is adapted to be mounted on one of a pole or a wall. The at least one ultraviolet light source and light reflector are disposed within the housing. The light reflector is adapted to reflect ultraviolet light waves generated by the ultraviolet light sources out of the housing. The motion detector or switch is supported by the housing and adapted to selectively energize the at least one ultraviolet light source to emit the ultraviolet light waves such that the ultraviolet light waves interact with hands of an individual placed under the housing to kill or deactivate harmful germs or transmissible diseases disposed on the hands of the individual, thereby preventing the harmful germs or transmissible diseases from spreading and causing harm to the individual or other individuals or animals.

In one embodiment, the ultraviolet light waves can comprise at least one of UV-C light waves, UV-B light waves, and UV-A light waves.

In another embodiment the device further comprises a second housing, a target surface, at least one second ultraviolet light source and second reflector, and a pole. The second housing is fixedly connected to the first housing. The target surface is of discrete dimensions and supported by the second housing and adapted to be stood upon by the individual. The at least one second ultraviolet light source and second light reflector is supported within the second housing and adapted to direct ultraviolet light waves generated by the at least one second ultraviolet light source onto the target surface such that the ultraviolet light waves interact with the feet of the individual standing upon the target surface, wherein the ultraviolet light waves kill or deactivate harmful germs or transmissible diseases disposed on the target surface, or carried by the feet or the socks of the individual, thereby preventing the harmful germs from spreading and causing harm to the individual or other individuals or animals. Finally, the pole connects the first housing to the second housing such that the device is freestanding.

In one embodiment, the second housing comprises a pair of channels disposed on opposite sides of the target surface for directing the ultraviolet light waves toward the target surface, and the at least one second ultraviolet light source and second light reflector disposed within the channels.

So configured, the channels include elongated openings adjacent the target surface.

In one embodiment, the target surface comprises a removable grate to enable the removal of trapped debris from beneath the target surface.

In another embodiment, the device further comprises a means for moving air through the target surface and adjacent the at least one second ultraviolet light source to kill or deactivate air born germs or transmissible diseases released in the area of the target surface.

In still another embodiment, the device further comprises a means for emitting a germicidal chemical in the area of the target surface to kill or deactivate harmful germs or transmissible diseases. The germicidal chemical can comprise an antibiotic chemical.

According to another embodiment of the present disclosure, a freestanding device comprises a first housing, at least one first ultraviolet light source and first light reflector, one of a first motion detector or a first switch, a second housing, a target surface, at least one second ultraviolet light source and second light reflector, and a back support.

In this embodiment, the first housing is adapted to be supported by a floor. The at least one first ultraviolet light source and first light reflector are disposed within the first housing. The first reflector is adapted to reflect ultraviolet light waves generated by the at least one first ultraviolet light source out of the first housing. The first motion detector or first switch is supported by the first housing and adapted to selectively energize the at least one ultraviolet light source to emit the ultraviolet light waves such that the ultraviolet light waves interact with hands of an individual placed under the housing to kill or deactivate harmful germs or transmissible diseases disposed on the hands of the individual, thereby preventing the harmful germs or transmissible diseases from spreading and causing harm to the individual or other individuals or animals.

Moreover, the second housing is fixedly connected to the first housing. The target surface is of discrete dimensions, supported by the second housing, and adapted to be stood upon by the individual. The at least one second ultraviolet light source and second light reflector are supported within the second housing and adapted to direct ultraviolet light waves generated by the at least one second ultraviolet light source onto the target surface such that the ultraviolet light waves interact with the feet of the individual standing upon the target surface, wherein the ultraviolet light waves kill or deactivate harmful germs or transmissible diseases disposed on the target surface, or carried by the feet or the socks of the individual, thereby preventing the harmful germs from spreading and causing harm to the individual or other individuals or animals.

Finally, the back support connects the first housing to the second housing such that the device is freestanding.

In another embodiment, the device further comprises one of a second motion detector or a second switch supported by the second housing for selectively activating and deactivating the at least one second ultraviolet light source disposed within the second housing.

In yet another embodiment, the device further comprises a means for moving air through the target surface and adjacent the at least one second ultraviolet light source to kill or deactivate air born germs or transmissible diseases released in the area of the target surface.

In still another embodiment, the device further comprises a means for emitting a germicidal chemical in the area of the target surface to kill or deactivate harmful germs or transmissible diseases. The germicidal chemical can comprise an antibiotic chemical.

In yet another embodiment of the present disclosure, a device comprises a first housing, a first ultraviolet light source and first light reflector, one of a motion detector or a switch, a second housing, a target surface, a second ultraviolet light source and second light reflector, a third ultraviolet light source and third light reflector, and a back support.

The first housing comprises a hood with an opening on the bottom and a back plate for mounting on one of a wall or a back support. The first ultraviolet light source and first light reflector are disposed within the first housing. The first reflector is adapted to reflect ultraviolet light waves generated by the first ultraviolet light source out of the opening in the first housing. The motion detector or switch s supported by the first housing for selectively activating the first ultraviolet light source. The second housing defines first and second channels extending along opposite sides thereof. The target surface is of discrete dimensions, supported between the channels of the second housing, and adapted to be stood upon by an individual.

The second ultraviolet light source and second light reflector are supported by the second housing within the first channel. The third ultraviolet light source and third light reflector are supported by the second housing within the second channel, wherein the first and second channels of the second housing are shaped and configured with the second and third light reflectors to reflect ultraviolet light waves generated by the second and third ultraviolet light sources onto the target surface such that the ultraviolet light waves interact with the feet of the individual standing upon the target surface to kill or deactivate harmful germs or transmissible diseases disposed on the target surface, or carried by the feet or socks of the individual, thereby preventing the harmful germs or transmissible diseases from spreading and causing harm to other individuals or animals. The back support connects the first housing to the second housing to define a freestanding device.

In one embodiment, the first and second channels of the second housing define elongated openings extending the length and/or the width of the target surface.

In another embodiment, the device further comprises a means for moving air through the target surface and into the first and second channels of the second housing adjacent to the second and third ultraviolet light sources to kill or deactivate air born germs or transmissible diseases released in the area of the target surface.

In yet another embodiment, the target surface comprises a porous surface and the means for circulating air through the target surface comprises a fan for drawing air downward through the target surface.

In still another embodiment, the device further comprises a means for emitting a germicidal chemical in the area of the target surface to kill or deactivate harmful germs. The germicidal chemical can comprise an antibiotic chemical.

In one embodiment, the ultraviolet light waves comprise at least one of UV-C light waves, UV-B light waves, and UV-A light waves.

In one embodiment, the target surface comprises a removable grate supported by the second housing to enable the removal of trapped debris from beneath the target surface.

A still further embodiment of the present disclosure includes a method of killing or deactivating harmful germs or transmissible diseases carried by hands or feet of an individual. According to one embodiment, the method comprises detecting the presence of the hands of the individual in the proximity of a hand cleaning device equipped with one of a motion detector and a switch and supported by a back support. Then, generating first ultraviolet light waves with one or more first ultraviolet light sources disposed within the hand cleaning device. Then, deflecting the first ultraviolet light waves out of an opening in the hand cleaning device toward the hands of the individual such that the first ultraviolet light waves interact with the hands to kill or deactivate harmful germs or transmissible diseases disposed on the hands of the individual. Then generating second ultraviolet light waves with one or more second ultraviolet light sources disposed within channels disposed on opposite sides of a target surface of a foot cleaning device. Then deflecting the second ultraviolet light waves out of the channels and toward the target surface such that the second ultraviolet light waves interact with at least one of the target surface or the feet of the individual walking across the target surface to kill or deactivate harmful germs or transmissible diseases disposed on the target surface or carried by the feet of the individual. Then, finally, performing at least one of the following: (a) circulating air through the target surface of the foot cleaning device toward the one or more second ultraviolet light sources disposed within the channels to kill or deactivate air born germs or transmissible diseases released in the area of the target surface; and (b) emitting a germicidal chemical in the area of the target surface to kill or deactivate harmful germs or transmissible diseases.

Accordingly, the present disclosure provides a hand cleaning system including a wall-mounted hand unit and germicidal floor and hand cleaning system as a freestanding device, or comprised of both germicidal floor unit and hand cleaning unit in one body that utilize ultraviolet light to effectively eradicate, deactivate, kill and/or compromise germs on hands and feet with or without the aid of soap and water, antibiotic hand gel or other chemicals. Such a system can be installed in health/medical facilities, sports facilities and gyms, homes, bathrooms and kitchen, restaurants, and other public area suspected of conduit for transmitting germs.

### BRIEF DESCRIPTION OF THE DRAWINGS

The following drawings are representative ideas and designs of various systems of the present disclosure. The systems are designed to protect the devices from impact damage. As one can clearly see, the devices can be of many different designs.

FIG. 1 is a perspective view of one embodiment of a wall-mountable hand cleaning unit constructed in accordance with the principles of the present disclosure;

FIG. 2 is a perspective view of one embodiment of a freestanding hand cleaning unit constructed in accordance with the principles of the present disclosure;

FIG. 3 is a perspective view of one embodiment of a freestanding, combination hand cleaning unit and foot cleaning unit constructed in accordance with the principles of the present disclosure;

FIG. 4 is a perspective view of another embodiment of a freestanding, combination hand cleaning unit and foot cleaning unit constructed in accordance with the principles of the present disclosure;

FIG. 5 is a perspective view of one embodiment of a foot cleaning unit constructed in accordance with the principles of the present disclosure;

FIG. 6 is a side view of the foot unit of FIG. 5; and

FIG. 7 is a side view of the foot unit of FIGS. 5 and 6 shown in use.

### GENERAL DESCRIPTION OF THE DISCLOSURE

One of the most germ-infested places is sports facility, in particular the sports equipment and the floor. A warm environment full of sweating bodies and exposed skin makes the gym a haven for bacteria, viruses, parasites, mold and fungi growth. It is well known and anticipated that shared sports equipment in the facilities are full of germs that body parts that come in contact with these equipment can be conduit for a cause of major health problem(s).

When people take off their shoes after exercising in a gym, they run into possibilities of transmitting contagious germs from one person to another or several individuals. These problems culminate because the sweaty environment inside the shoes is most ideal for these germs to grow multiply. Wherever people take off their shoes and walk in socks or bare feet on common area such as home, gym, swimming pool areas, athletic facility locker rooms and etc., the germs are now contaminating everyone else who comes in contact.

In the past few years, resistant staphylococcus, called methicillin-resistant Staphylococcus aureus (MRSA), is becoming increasingly common in athletes participating in indoor sports. This invention is designed to stop the spreading or transmission of disease causing or harmful microorganisms (bacteria, virus, parasites, fungi, mold and spores thereof) to individuals at health/medical facilities, sports centers and gyms, homes, bathrooms and kitchen, restaurants, and other public area suspected of conduit for transmitting germs.

This disclosure relates to specially designed germicidal device(s) that are designed to eradicate, kill, or compromise the potentially pathologic germs using germicidal ultraviolet lights (UV-C) alone and/or other UV lights such as UV-A and Uv-B and/or in combination with other device(s) that will deactivate or render harmless the disease causing germs and/or spores thereof on hand(s) and feet.

This disclosure also relates to germicidal devices that are designed to protect individuals from contracting diseases or transmitting diseases through physical contact with germ contaminated floor and/or equipment at sports centers and gyms, homes, bathrooms and kitchen, and other public area suspected of conduit for transmitting germs. The invention further provides means to stop the spreading or transmission of disease causing or harmful microorganisms or spores thereof from infected individuals to other individuals. Specifically the invention provides devices that use germicidal ultraviolet light to kill or deactivate harmful germs (mold, fungi, bacteria, viruses) on hand(s) and feet that come in contact with equipment and floor of sports centers and gyms, homes, bathrooms and kitchen, and other public area suspected of conduit for transmitting germs.

The disclosure relates to devices that will kill or deactivate not only one or two germs that cause diseases but an array of all known and yet to be discovered disease causing microorganisms using ultraviolet light (UV-C) alone.

The disease causing microorganisms is not limited to those deactivated or killed by germicidal UV-C alone but also in combination with other ultraviolet lights such as UV-A and UV-B or in combinations ultraviolet lights thereof.

This disclosure relates to wall-mounted hand unit and freestanding device(s) comprised of both a germicidal floor unit and a hand cleaning unit in one body that house germicidal UV-C light source(s) and/or in combination with UV-A and/or UV-B light source(s) that also includes electrical components such as ballasts, sockets, a motion detector, and switch(s) for electric power connection.

This disclosure relates to wall mount hand unit and freestanding device(s) comprised of both a germicidal floor unit and a hand cleaning unit in one body of various sizes and shapes made out of different material such as plastics, fiber glasses, graphite, metals etc, and/or in combinations thereof, with a unit that house germicidal UV-C light source(s) of various power (wattages) and/or in combination with UV-A and/or UV-B light source(s) of various sizes (inches to several feet) to apply effective germicidal function at sports centers and gyms, homes, bathrooms and kitchen, and other public area suspected of conduit for transmitting germs.

One aspect of the disclosure is directed to wall-mounted hand unit, foot unit, and freestanding device(s) comprised of both a germicidal floor unit and a hand cleaning unit in one body that are designed to protect individuals from contracting or transmitting diseases through contact with equipment and floor of health/medical facilities, sports centers and gyms, homes, bathrooms and kitchen, restaurants, and other public area suspected of conduit for transmitting germs.

One aspect of the disclosure is to provide protection to people who run into possibilities of contracting germs because they come in physical contact with equipment and floor of health/medical facilities, sports centers and gyms, homes, bathrooms and kitchen, restaurants, and other public area suspected of conduit for transmitting germs.

Another aspect of the disclosure provides wall-mounted hand unit, foot unit, and freestanding device(s) comprised of both a germicidal floor unit and a hand cleaning unit in one body that use germicidal ultraviolet light to kill or deactivate harmful germs (mold, fungi, bacteria, parasites, viruses, and spores thereof) from spreading and causing harms to other individuals that come in contact.

Another aspect of the disclosure provides wall-mounted hand unit, foot unit, and freestanding device(s) comprised of both a germicidal floor unit and a hand cleaning unit in one body that use germicidal ultraviolet light to kill or deactivate harmful germs (mold, fungi, bacteria, parasites, viruses, and spores thereof) on hand(s) and feet with or without the aid of soap and water, antibiotic hand gel or other chemicals.

Another aspect of the disclosure is not only limited to using germicidal ultraviolet lights alone but also in combination with chemicals such as antibiotics or chemicals with germicidal properties that in contact with germs will kill or deactivate the microorganisms or spores thereof.

Another aspect of the disclosure relates to wall-mounted hand unit, foot unit, and freestanding device(s) comprised of both a germicidal floor unit and a hand cleaning unit in one body that will kill or deactivate not only one or two germs that cause diseases but an array of all known and yet to be discovered disease causing microorganisms using germicidal ultraviolet light (UV-C) alone.

Another aspect of the disclosure relates to wall-mounted hand unit, foot unit, and freestanding device(s) comprised of both a germicidal floor unit and a hand cleaning unit in one body that will kill or deactivate not only one or two germs that cause diseases but an array of all known and yet to be discovered disease causing microorganisms using germicidal ultraviolet light (UV-C) alone and/or in combination with germicidal chemicals.

Another aspect of the disclosure relates to disease causing microorganisms is not limited to those deactivated or killed by germicidal UV-C alone but also other ultraviolet lights such as UV-A and UV-B or in combinations of ultraviolet lights thereof.

Another aspect of the disclosure relates to disease causing germs not limited to those that are deactivated or killed by germicidal UV-C alone but also other ultraviolet lights such as UV-A and UV-B and also those that are further deactivated or killed by germicidal chemicals.

One aspect of the present disclosure is directed to wall-mounted hand unit, foot unit, and freestanding device(s) comprised of both a germicidal floor unit and a hand cleaning unit in one body that are designed to use various sizes of the ultraviolet light sources.

Another aspect of the disclosure is directed to wall-mounted hand unit, foot unit, and freestanding device(s) comprised of both a germicidal floor unit and a hand cleaning unit in one body that are designed to use various numbers and sizes of the ultraviolet light sources.

Another aspect of the disclosure is directed to wall-mounted hand unit, foot unit, and freestanding device(s) comprised of both a germicidal floor unit and a hand cleaning unit in one body that are designed to use various sizes and strength (wattage) of the ultraviolet light sources.

Another aspect of the disclosure is directed to wall-mounted hand unit, foot unit, and freestanding device(s) comprised of both a germicidal floor unit and a hand cleaning unit in one body that are designed to use various numbers and sizes and strength (wattage) of the ultraviolet light sources.

Another aspect of the disclosure is directed to wall-mounted hand unit, foot unit, and freestanding device(s) comprised of both a germicidal floor unit and a hand cleaning unit in one body that are designed to easily replace the ultraviolet light sources and other electronic components when device malfunction occurs.

Another aspect of the disclosure is directed to wall-mounted hand unit, foot unit, and freestanding device(s) comprised of both a germicidal floor unit and a hand cleaning unit in one body that are designed to protect the light bulb and other electronic parts from impact damage.

One aspect of the disclosure is directed to wall-mounted hand unit, foot unit, and freestanding device(s) comprised of both a germicidal floor unit and a hand cleaning unit in one body that are of different width and length to accommodate the requirement and needs of health/medical facilities, sports centers and gyms, homes, bathrooms and kitchen, restaurants, and other public area suspected of conduit for transmitting germs to be effective and cost efficient.

Another aspect of the disclosure is directed to wall-mounted hand unit, foot unit, and freestanding device(s) comprised of both a germicidal floor unit and a hand cleaning unit in one body that are made of various materials but not limited to plastics, fiberglass, graphite, metals and etc that house electrical components and UV light bulbs, switches and other necessary components with different width and length to be hand-held units or stand alone hand-push wheels on the bottom units.

### DETAILED DESCRIPTION OF THE PREFERRED EMBODIMENTS

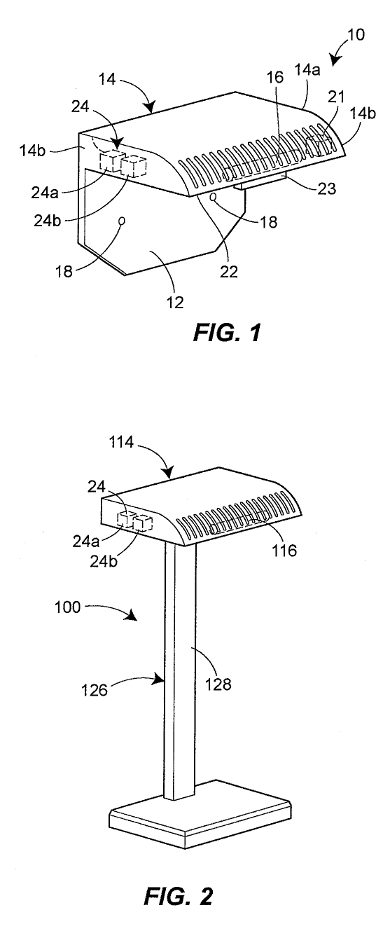

Referring now to the figures, FIG. 1 depicts one embodiment of a hand cleaning unit 10 constructed in accordance with the principles of the present disclosure. The hand cleaning unit 10 is adapted to be mounted to a wall or other freestanding structure. The hand cleaning unit 10 generally includes a support plate 12, a hood 14, and an ultraviolet light source 16. The support plate 12 and hood 14 can be referred to collectively as a housing, for example. The support plate 12 is a generally flat plate and can include one or more holes 18 for receiving fasteners such as screws, for example, for mounting the hand cleaning unit 10 to a wall. The hood 14 includes a slightly curved top plate 14a and a pair of side plates 14b.

So configured, the hood 14 defines a cavity 20 and an opening 22. The cavity 20 is adapted to accommodate the ultraviolet light source 16, as illustrated. In one embodiment, either or both of the top plate 14a and side plates 14b can include a reflector or reflective material adapted to reflect the light waves generated by the ultraviolet light source 16 out of the opening 22. In the present embodiment, the opening 22 is generally directed downward when the hand cleaning unit 10 is mounted to a wall. The ultraviolet light source 16 can generally include an elongated ultraviolet light bulb mounted within a ballast supported within the hood 14. The ballast would include a power chord (not shown) extending from the hand cleaning unit 10 to be either plugged into an electrical outlet or hard-wired to an electrical breaker, for example.

Additionally, as depicted in FIG. 1, one embodiment of a hand cleaning device 10 constructed in accordance with the principles of the present disclosure includes an electronic control unit 24. The electronic control unit 24 can be equipped with a motion detector and/or switch 24a and a timer 24b. So configured, the motion detector and/or switch 24a can detect when a user moves his/her hands beneath the opening 22 of the hood 14. For example, when the motion detector and/or switch 24a comprises simply a motion detector, the motion detector and/or switch 24a automatically detects the presence of the hands. When the motion detector and/or switch 24a comprises a switch, the switch can include either a manual switch that must be manipulated by the user, or an automatic switch that does not have to be manipulate by the user.

When the hands are present, the motion detector and/or switch 24a generates a signal to energize the ultraviolet light source 16. Additionally, the motion detector and/or switch 24a activates the timer 24b when a user's hands are present such that the electronic control unit 24 can de-energize the ultraviolet light source 16 upon the passing of a predetermined amount of time. For example, depending on the specific application and environment of use for the hand cleaning unit 10, the timer 24b can allow the ultraviolet light source 16 to be energized for 30 seconds. Such a configuration would signal to the user that his/her hands are completely cleaned by the hand cleaning unit 10 by turning off the ultraviolet light source 16.

Still referring to FIG. 1, the hand cleaning unit 10 can be equipped with a means for moving air, e.g., a fan 21, disposed within the cavity 20 of the hood 14. The fan 21 can be activated to circulate air in the general vicinity of the hand cleaning unit 10 over the ultraviolet light source 16, thereby killing or deactivating any harmful air born germs, or transmissible diseases. Furthermore, one embodiment of the hand cleaning device 10 can include a means for emitting a germicidal chemical 23 mounted within the hood 23. The means for emitting a germicidal chemical 23 can include a sprayer, for example, adapted to emit a germicidal chemical in the area of the hand cleaning unit 10 to kill or deactivate harmful germs. The germicidal chemical can include an antibiotic chemical, for example, carried within a hand gel, soap, or other carrier.

FIG. 2 depicts an alternative hand cleaning unit 100 constructed in accordance with the teachings of the present disclosure. The hand cleaning unit 100 comprises a free-standing unit and thus, does not need to be mounted to a wall or other support structure. The hand cleaning unit 100 includes a hood 114, a stand 126, and an ultraviolet light source 116. The hood 114 is identical to the hood 14 depicted in FIG. 1 and accommodates the light source 116 in a manner identical to that described above. The hood 114 can also be referred to as a housing for the hand cleaning unit 100. Moreover, the hood 114 includes an electronic control unit 124 that is generally identical to the electronic control unit 24 described above for activating and deactivating the hand cleaning unit 100. That is, the electronic control unit 124 can include a motion detector and/or switch 124a and a timer 124b. In another embodiment, the hood 114 can also be equipped with means for moving air and means for emitting germicidal chemicals as described above.

The stand 126 of the hand cleaning unit 100 depicted in FIG. 2 includes a back support, which in this embodiment includes a pole 128 and a foot plate 130. The pole 128 is an elongated pole and can include a hollow pipe, channel, or other structure. The foot plate 130 is a generally flat plate of appropriate dimensions to suitably prevent the hand cleaning device 100 from tipping over.

While the hand cleaning device 100 has been described as including an electronic control unit 124 including a motion detector and/or switch 124a for activating the ultraviolet light source 116, in another embodiment, the foot plate 130 can include a pressure sensor, pedal, or other device. So configured, a user can step on the pressure sensor, pedal, or other device to activate the ultraviolet light source 116, and optionally, the means for moving air and means for emitting a germicidal chemical.

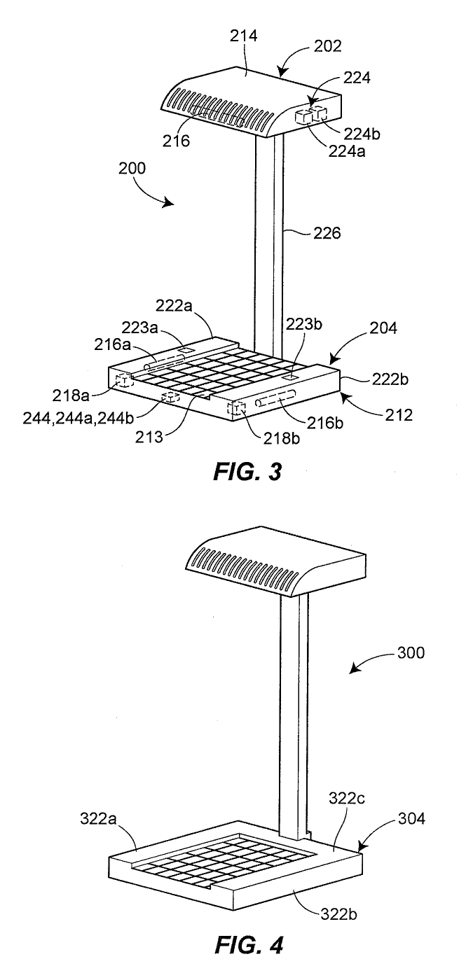

FIG. 3 depicts yet another embodiment of a device 200 constructed in accordance with the principles of the present disclosure. The device 200 generally comprises a freestanding device having a hand cleaning unit 202 supported above a foot cleaning unit 204 on a back support, which in this embodiment includes a pole 226. The hand cleaning unit 202 can be generally identical to the hand cleaning units 10, 100 described above with reference to FIGS. 1 and 2. For example, the hand cleaning unit 202 generally includes a hood 214, an ultraviolet light source 216, and an electronic control unit 224 with motion detector and/or switch 224a and timer 224b.

The foot cleaning unit 204 can include generally any foot cleaning unit and, preferably can include a foot cleaning unit generally identical to one of the foot cleaning units described in U.S. patent application Ser. No. 12/101,494, entitled “Germicidal Floor System (GFS),” filed Apr. 11, 2008, the entire contents of which are expressly incorporated herein by reference.

For example, the foot cleaning unit 204 generally includes a housing 212, a grate 213, a plurality of ultraviolet light sources 216a, 216b, and two means for moving air 218a, 218b. The housing 212 defines a pair of channels 222a, 222b accommodating the light sources 216a, 216b and the means for moving air 218a, 218b. The grate 213 is supported between the channels 222a, 222b and is adapted to be stood on by a user. So configured, the channels 222a, 222b include reflective surfaces defining reflectors for reflecting light waves generated by the light sources 216a, 216b toward the grate 213, the top surface of which defines a target surface. Moreover, the means for moving air 218a, 218b can include fans for drawing air through the grate 213 and back into the channels 222a, 222b adjacent the light sources 216a, 216b to kill or deactivate any harmful air born germs or transmissible diseases in the vicinity of the grate 213. In another embodiment, the channels 222a, 222b can also include means for emitting germicidal chemicals 223a, 223b, such as antibiotic chemicals, in the region of the grate 213. The remaining details of the foot cleaning unit 204 are generally identical to those described in U.S. patent application Ser. No. 12/101,494, and therefore, will not be repeated herein.

Still referring to FIG. 3, the foot cleaning unit 204 can include an electronic control unit 244 similar to the electronic control unit 224 in the hand cleaning unit 202. The electronic control unit 244 can include a pressure sensor 244a and a timer 244b. So configured, the pressure sensor 244a detects when a user stands on the foot cleaning unit 204. Once a standing user is detected, the electronic control unit 244 can activate the light sources 216a, 216b, the means for moving air 218a, 218b, and/or the means for emitting germicidal chemicals 223a, 223b. The timer 244b then deactivates the light sources 216a, 216b, the means for moving air 218a, 218b, and/or the means for emitting germicidal chemicals 223a, 223b after some predetermined period of time. In an alternative embodiment, the electronic control unit 224 of the foot cleaning unit 204 can include a motion sensor instead of the pressure sensor 244a.

The foot cleaning unit 204 depicted in FIG. 3 includes two channels 222a, 222b arranged along opposite side edges of the housing 212, and therefore, can be referred to as a two-channel foot cleaning unit.

FIG. 4 depicts an alternative embodiment of a device 300 that is generally identical to the device 200 depicted in FIG. 3, but includes what can be referred to as a three-channel foot cleaning unit 304. That is, the foot cleaning unit 304 depicted in FIG. 4 includes two opposing side channels 322a, 322b, as well as a transverse front channel 322c. Although not expressly depicted, in this embodiment, each of the three channels 322a, 322b, 322c can be equipped with ultraviolet light sources and/or means for moving air. So configured, the foot cleaning device 304 provides a different structural configuration from that described above, which can provide a different cleaning function.

While the freestanding devices 200, 300 depicted in FIGS. 3 and 4 have been described as including two-channel and three-channel foot cleaning devices 204, 304, respectively, alternative devices can include alternative foot cleaning devices.

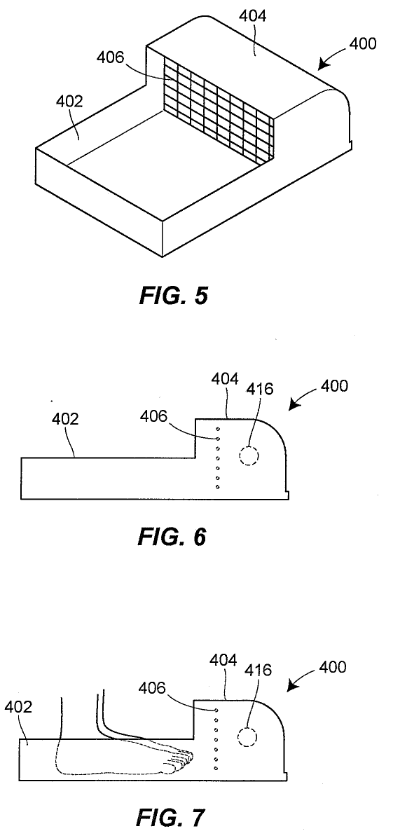

For example, FIGS. 5-7 depict one alternative foot cleaning device 400 that could be incorporated with either of the devices 200, 300 described above with reference to FIGS. 3 and 4. The foot cleaning device 400 generally includes a tray portion 402, a hood portion 404, and a grate 406. The tray portion 402 is adapted to accommodate a user's feet. The hood portion 404 is adapted to accommodate an ultraviolet light source 416 for cleaning the user's feet. Additionally, in one embodiment, the hood portion 404 can accommodate a means for moving air similar to the means for moving air described in U.S. patent application Ser. No. 12/101,494 mentioned above. So configured, the foot cleaning device 400 can clean a user's feet in the manner described herein.

Although not discussed above, the freestanding cleaning units 100, 200, 300 of FIGS. 2, 3, and 4 can stand in the range of approximately 3 feet (0.9 meters) to approximately 5 feet (1.5 meters) in height. However, this is just an example and it should be appreciated that other heights are intended to fall within the scope of the disclosure.

In light of the foregoing, the present disclosure could generally be stated as comprising a wall-mounted hand unit, foot unit, and/or freestanding device comprised of both a germicidal floor unit and a hand cleaning unit in one body that will protect individuals from contracting or transmitting disease causing germs (bacteria, fungi, viruses, parasites, mold and spores thereof) by contact with equipment and floor of health/medical facilities, sports centers and gyms, homes, bathrooms and kitchen, restaurants, and other public area suspected of conduit for transmitting germs.

Additionally, the present disclosure could generally be stated as comprising a wall-mounted hand unit, foot unit, and/or freestanding device comprised of both a germicidal floor unit and a hand cleaning unit in one body that are designed to protect individuals who run into possibilities of contracting and/or transmitting germs from one person to another or several individuals because they need to put on and/or take off their shoes or barefoot or come in contact with equipment and floor of health/medical facilities, sports centers and gyms, homes, bathrooms and kitchen, restaurants, and other public area suspected of conduit for transmitting germs.

Additionally, the present disclosure could generally be stated as comprising a wall-mounted hand unit, foot unit, and/or freestanding devices comprised of both a germicidal floor unit and a hand cleaning unit in one body that are designed to protect individuals who run into possibilities of contracting germs from one person to another or several individuals from health/medical facilities, exercise mats of sports facilities and gyms and related thereof because they need to put on and/or take off their shoes and walk on a common area with foot traffic, either barefoot or with socks on.

Additionally, the present disclosure could generally be stated as comprising a wall-mounted hand unit, foot unit, and/or freestanding device comprised of both a germicidal floor unit and a hand cleaning unit in one body that are designed to use germicidal ultraviolet light to kill or deactivate harmful germs (mold, fungi, bacteria, viruses, parasitic worms, etc) from spreading and causing harms to people or animals that come in contact with equipment and floor of health/medical facilities, sports centers and gyms, homes, bathrooms and kitchen, restaurants, and other public area suspected of conduit for transmitting germs.

Additionally, the present disclosure could generally be stated as comprising a wall-mounted hand unit, foot unit, and/or a freestanding device comprised of both a germicidal floor unit and a hand cleaning unit in one body that are designed to use germicidal ultraviolet light to kill or deactivate harmful germs (mold, fungi, bacteria, viruses, parasitic worms, etc) on hand(s) and feet with or without the aid of soap and water, antibiotic hand gel or other chemicals to prevent/protect spreading and causing harms to people or animals that come in contact.

Additionally, the present disclosure could generally be stated as comprising a wall-mounted hand unit, foot unit, and/or a freestanding device comprised of both a germicidal floor unit and a hand cleaning unit in one body that are designed not only limited to using germicidal ultraviolet lights alone but also in combination with chemicals such as antibiotics or chemicals with germicidal properties that in contact with germs will kill or deactivate the microorganisms or spores thereof.

Additionally, the present disclosure could generally be stated as comprising a wall-mounted hand unit, foot unit, and/or a freestanding device comprised of both a germicidal floor unit and a hand cleaning unit in one body that are designed to kill or deactivate not only one or two germs that cause diseases but an array of all known and yet to be discovered disease causing microorganisms using ultraviolet light (UV-C) alone.

Additionally, the present disclosure could generally be stated as comprising a wall-mounted hand unit, foot unit, and/or a freestanding device comprised of both a germicidal floor unit and a hand cleaning unit in one body that are designed to kill or deactivate not only one or two germs that cause diseases but an array of all known and yet to be discovered disease causing microorganisms using ultraviolet light (UV-C) alone and/or in combination with other UV lights such as UV-A and UV-B.

Additionally, the present disclosure could generally be stated as comprising a wall-mounted hand unit, foot unit, and/or a freestanding device comprised of both a germicidal floor unit and a hand cleaning unit in one body with built in ventilation fan(s) to control the light sources (light bulbs) from getting overheated that are designed to kill or deactivate germs that cause diseases using ultraviolet light (UV-C) alone and/or in combination with other UV lights such as UV-A and UV-B.

Additionally, the present disclosure could generally be stated as comprising a wall-mounted hand unit, foot unit, and/or a freestanding device comprised of both a germicidal floor unit and a hand cleaning unit in one body that are designed to kill or deactivate not only one or two germs that cause diseases but an array of all known and yet to be discovered disease causing microorganisms using ultraviolet light (UV-C) alone and/or in combination with other UV lights such as UV-A and UV-B and/or in combination with germicidal chemicals.

Additionally, the present disclosure could generally be stated as comprising a wall-mounted hand unit, foot unit, and/or a freestanding device comprised of both a germicidal floor unit and a hand cleaning unit in one body that will protect individuals from transmissible disease causing germs and/or spores thereof by coming in contact with equipment and floor of health/medical facilities, sports centers and gyms, homes, bathrooms and kitchen, restaurants, and other public area suspected of conduit for transmitting germs using germicidal ultraviolet light (UV-C) that will irradiate hand(s) and feet with or without the aid of soap and water, antibiotic hand gel or other chemicals.

Additionally, the present disclosure could generally be stated as comprising a wall-mounted hand unit, foot unit, and/or a freestanding device comprised of both a germicidal floor unit and a hand cleaning unit in one body that will protect individuals from transmissible disease causing germs and/or spores thereof by coming in contact with equipment and floor of health/medical facilities, sports centers and gyms, homes, bathrooms and kitchen, restaurants, and other public area suspected of conduit for transmitting germs using germicidal ultraviolet light (UV-C) and/or in combination of other UV lights such as UV-A and UV-B and/or with germicidal chemicals on hand(s) and feet with or without the aid of soap and water, antibiotic hand gel or other chemicals.

Additionally, the present disclosure could generally be stated as comprising a wall-mounted hand unit, foot unit, and/or a freestanding device comprised of both a germicidal floor unit and a hand cleaning unit in one body that will protect individuals from transmissible disease causing germs and/or spores thereof by coming in contact with equipment and floor of health/medical facilities, sports centers and gyms, homes, bathrooms and kitchen, restaurants, and other public area suspected of conduit for transmitting germs using germicidal ultraviolet light (UV-C) and/or in combination of other UV lights such as UV-A and UV-B and/or with germicidal chemicals on hand(s) and feet with or without the aid of soap and water, antibiotic hand gel or other chemicals; whereas the UV light source(s) and other electronic components such as ventilation fans, ballasts, and sockets are easily replaced when in need of replacement due to malfunction (a state of breakage and/or the light bulb and/or ventilation fan(s) going out).

Furthermore, the present disclosure can be generally described as comprising a method of stopping the spreading or transmission of disease causing or harmful microorganisms (mold, fungi, bacteria, viruses, parasites, and spores thereof) from infected individuals to other individuals through contact with equipment and floor of health/medical facilities, sports centers and gyms, homes, bathrooms and kitchen, restaurants, and other public area suspected of conduit for transmitting germs.

Furthermore, the present disclosure can be generally described as comprising a method of protecting individuals from transmissible disease causing germs (viruses, bacteria, fungi, parasitic worms, mold, and/or spores thereof) from contact with germs on the surface of equipment and floor of health/medical facilities, sports centers and gyms, homes, bathrooms and kitchen, restaurants, and other public area suspected of conduit for transmitting germs.

Furthermore, the present disclosure can be generally described as comprising a method of protecting individuals from transmissible disease causing germs and/or spores thereof using germicidal ultraviolet light (UV-C) and/or in combination with other UV lights such as UV-A and UV-B, with/without application of germicidal chemicals, irradiating hand(s) and feet that came in contact with the equipment and floor of health/medical facilities, sports centers and gyms, homes, bathrooms and kitchen, restaurants, and other public area suspected of conduit for transmitting germs.

Further still, the present disclosure can generally be states as comprising a wall-mounted hand unit, foot unit, and/or freestanding device comprised of both a germicidal floor unit and a hand cleaning unit in one body that are designed to use various numbers and sizes and strength (wattage) of the ultraviolet light sources.

Further still, the present disclosure can generally be states as comprising a wall-mounted hand unit, foot unit, and/or freestanding device comprised of both a germicidal floor unit and a hand cleaning unit in one body that is of different width and length to accommodate the requirement of health/medical facilities, sports centers and gyms, homes, bathrooms and kitchen, restaurants, and other public area suspected of conduit for transmitting germs but not limited to these applications but else where who is skilled in art can perceive this invention can be applied.

Further still, the present disclosure can generally be states as comprising a wall-mounted hand unit, foot unit, and/or freestanding device comprised of both a germicidal floor unit and a hand cleaning unit in one body that are made of various materials but not limited to plastics, fiberglass, graphite, metals and etc that house electrical components, UV light bulbs, switches, motion detector and/or switch, and other necessary components with different width and length.

Further still, the present disclosure can generally be states as comprising a wall-mounted hand unit, foot unit, and/or freestanding device comprised of both a germicidal floor unit and a hand cleaning unit in one body that are made of various materials but not limited to plastics, fiberglass, graphite, metals and etc that house electrical components and UV light bulbs, switches and other necessary components and reflector made of mirror(s) or shiny metallic material that will reflect the UV radiation to maximize the UV irradiation and minimize the loss of UV radiation with different width and length to accommodate the units.

This invention is not limited to what is described herein but rather is also inclusive of similar, equivalent, and derivative technology, designs, methods of use, and applications that are obvious to those versed in the art.

What is claimed is:

1. A device, comprising:
a first housing adapted to be mounted on one of a pole or a wall;
at least one ultraviolet light source and a light reflector disposed within the housing, the light reflector adapted to reflect ultraviolet light waves generated by the ultraviolet light sources out of the housing; and
one of a motion detector or a switch supported by the housing and adapted to selectively energize the at least one ultraviolet light source to emit the ultraviolet light waves such that the ultraviolet light waves interact with hands of an individual placed under the housing to kill or deactivate harmful germs or transmissible diseases disposed on the hands of the individual, thereby preventing the harmful germs or transmissible diseases from spreading and causing harm to the individual or other individuals or animals.

2. The device of claim 1, wherein the ultraviolet light waves comprise at least one of UV-C light waves, UV-B light waves, and UV-A light waves.

3. The device of claim 1, further comprising:
a second housing fixedly connected to the first housing;
a target surface of discrete dimensions supported by the second housing and adapted to be stood upon by the individual;
at least one second ultraviolet light source and second light reflector supported within the second housing and adapted to direct ultraviolet light waves generated by the at least one second ultraviolet light source onto the target surface such that the ultraviolet light waves interact with the feet of the individual standing upon the target surface, wherein the ultraviolet light waves kill or deactivate harmful germs or transmissible diseases disposed on the target surface, or carried by the feet or the socks of the individual, thereby preventing the harmful germs from spreading and causing harm to the individual or other individuals or animals; and
a pole connecting the first housing to the second housing such that the device is freestanding.

4. The device of claim 3, wherein the second housing comprises one or more channels disposed on sides of the target surface for directing the ultraviolet light waves toward the target surface, the at least one second ultraviolet light source and second light reflector disposed within the channels.

5. The device of claim 4, wherein the channels include elongated openings adjacent the target surface.

6. The device of claim 3, wherein the target surface comprises a removable grate to enable the removal of trapped debris from beneath the target surface.

7. The device of claim 3, further comprising a means for moving air through the target surface and adjacent the at least one second ultraviolet light source to kill or deactivate air born germs or transmissible diseases released in the area of the target surface.

8. The device of claim 3, further comprising a means for emitting a germicidal chemical in the area of the target surface to kill or deactivate harmful germs or transmissible diseases, wherein the germicidal chemical comprises an antibiotic chemical.

9. A freestanding device, comprising:
a first housing adapted to be supported by a floor; and
at least one first ultraviolet light source and first light reflector disposed within the first housing, the first reflector adapted to reflect ultraviolet light waves generated by the at least one first ultraviolet light source out of the first housing;
one of a first motion detector or a first switch supported by the first housing and adapted to selectively energize the at least one ultraviolet light source to emit the ultraviolet light waves such that the ultraviolet light waves interact with hands of an individual placed under the housing to kill or deactivate harmful germs or transmissible diseases disposed on the hands of the individual, thereby preventing the harmful germs or transmissible diseases from spreading and causing harm to the individual or other individuals or animals;
a second housing fixedly connected to the first housing;
a target surface of discrete dimensions supported by the second housing and adapted to be stood upon by the individual;
at least one second ultraviolet light source and second light reflector supported within the second housing and adapted to direct ultraviolet light waves generated by the at least one second ultraviolet light source onto the target surface such that the ultraviolet light waves interact with the feet of the individual standing upon the target surface, wherein the ultraviolet light waves kill or deactivate harmful germs or transmissible diseases disposed on the target surface, or carried by the feet or the socks of the individual, thereby preventing the harmful germs from spreading and causing harm to the individual or other individuals or animals; and
a back support connecting the first housing to the second housing such that the device is freestanding.

10. The device of claim 9, further comprising one of a second motion detector or a second switch supported by the second housing for selectively activating and deactivating the at least one second ultraviolet light source disposed within the second housing.

11. The device of claim 9, further comprising a means for moving air through the target surface and adjacent the at least one second ultraviolet light source to kill or deactivate air born germs or transmissible diseases released in the area of the target surface.

12. The device of claim 9, further comprising a means for emitting a germicidal chemical in the area of the target surface to kill or deactivate harmful germs or transmissible diseases, wherein the germicidal chemical comprises an antibiotic chemical.

13. A device, comprising:
a first housing comprising a hood with an opening on the bottom and a back plate for mounting on one of a wall or a back support;
a first ultraviolet light source and first light reflector disposed within the first housing, the first reflector adapted to reflect ultraviolet light waves generated by the first ultraviolet light source out of the opening in the first housing;
one of a motion detector or a switch supported by the first housing for selectively activating the first ultraviolet light source;
a second housing defining one or more channels extending along sides thereof;
a target surface of discrete dimensions supported between the channels of the second housing and adapted to be stood upon by an individual;
a second ultraviolet light source and second light reflector supported by the second housing within a first of the one or more channels;
a third ultraviolet light source and third light reflector supported by the second housing within a second of the one or more channels, wherein the one or more channels of the second housing are shaped and configured with the second and third light reflectors to reflect ultraviolet light waves generated by the second and third ultraviolet light sources onto the target surface such that the ultraviolet light waves interact with the feet of the individual standing upon the target surface to kill or deactivate harmful germs or transmissible diseases disposed on the target surface, or carried by the feet or socks of the individual, thereby preventing the harmful germs or transmissible diseases from spreading and causing harm to other individuals or animals; and
a back support that connects the first housing to the second housing to define a freestanding device.

14. The device of claim 13, wherein the one or more channels of the second housing define elongated openings extending the length and/or the width of the target surface.

15. The device of claim 13, further comprising a means for moving air through the target surface and into the one or more channels of the second housing adjacent to the second and third ultraviolet light sources to kill or deactivate air born germs or transmissible diseases released in the area of the target surface.

16. The device of claim 15, wherein the target surface comprises a porous surface and the means for circulating air through the target surface comprises a fan for drawing air downward through the target surface.

17. The device of claim 13, further comprising a means for emitting a germicidal chemical in the area of the target surface to kill or deactivate harmful germs, wherein the germicidal chemical comprise antibiotic chemicals.

18. The device of claim 13, wherein the ultraviolet light waves comprise at least one of UV-C light waves, UV-B light waves, and UV-A light waves.

19. The device of claim 13, wherein the target surface comprises a removable grate supported by the second housing to enable the removal of trapped debris from beneath the target surface.

20. A method of killing or deactivating harmful germs or transmissible diseases carried by hands or feet of an individual, the method comprising:
detecting the presence of the hands of the individual in the proximity of a hand cleaning device equipped with one of a motion detector and a switch and supported by a back support;
generating first ultraviolet light waves with one or more first ultraviolet light sources disposed within the hand cleaning device;
deflecting the first ultraviolet light waves out of an opening in the hand cleaning device toward the hands of the individual such that the first ultraviolet light waves interact with the hands to kill or deactivate harmful germs or transmissible diseases disposed on the hands of the individual;
generating second ultraviolet light waves with one or more second ultraviolet light sources disposed within one or more channels disposed on sides of a target surface of a foot cleaning device;
deflecting the second ultraviolet light waves out of the one or more channels and toward the target surface such that the second ultraviolet light waves interact with at least one of the target surface or the feet of the individual walking across the target surface to kill or deactivate harmful germs or transmissible diseases disposed on the target surface or carried by the feet of the individual; and
at least one of the following:
circulating air through the target surface of the foot cleaning device toward the one or more second ultraviolet light sources disposed within the one or more channels to kill or deactivate air born germs or transmissible diseases released in the area of the target surface; and
emitting a germicidal chemical in the area of the target surface to kill or deactivate harmful germs or transmissible diseases.
